(19) United States
(12) Patent Application Publication
(10) Pub. No.: US 2025/0381231 A1
(43) Pub. Date: Dec. 18, 2025

(54) METHODS RELATING TO TISSUE REGENERATION

(71) Applicant: Northwestern University, Evanston, IL (US)

(72) Inventors: Arun SHARMA, Evanston, IL (US); Mitall Kini, Evanston, IL (US); Matthew Bury, Evanston, IL (US)

(21) Appl. No.: 19/237,986

(22) Filed: Jun. 13, 2025

Related U.S. Application Data
(60) Provisional application No. 63/659,729, filed on Jun. 13, 2024.

(51) Int. Cl. A61K 35/28; A61P 13/10

(52) U.S. Cl. CPC A61K 35/28; A61P 13/10

(57) ABSTRACT

The present disclosure provides methods related to tissue regeneration using human mesenchymal stem cell (MSC)/CD34+ hematopoietic stem/progenitor cell (HSPC) co-cultured total conditioned media. In particular, the present disclosure provides novel methods for treating and/or preventing damage to, trauma to, and/or loss of tissue.

### CROSS-REFERENCE

This application claims the benefit of U.S. Provisional Patent Application No. 63/659,729, filed Jun. 13, 2024; which is incorporated by reference herein in its entirety.

### GOVERNMENT SUPPORT

This invention was made with government support under grant number DK109539 awarded by the National Institutes of Health. The government has certain rights in the invention.

### FIELD

The present disclosure provides methods related to tissue regeneration using total conditioned media (TCM) derived from the co-culture of human mesenchymal stem cells (MSC) with CD34+ hematopoietic stem/progenitor cells (HSPC). In particular, the present disclosure provides novel methods for treating and/or preventing damage to, trauma to, and/or loss of tissue.

### BACKGROUND

Impaired urinary bladder compliance, secondary to congenital or acquired bladder dysfunction, can lead to irreversible renal damage. Severe or end-stage bladder dysfunction is managed with surgical augmentation, utilizing intestinal tissue, which can, in part, contribute to increased stone formation, infections and malignant transformation. Co-seeded bone marrow derived mesenchymal stem cell (MSC)/CD34+ hematopoietic stem/progenitor cell (HSPC) scaffolds have been successful in regenerating bladder tissue. However, the acquisition of viable cells is challenging in the clinical setting. Cell-free total TCM methods, as an alternative to traditional cell-seeded scaffolds, are needed to promote bladder tissue regeneration.

### SUMMARY

Embodiments of the present disclosure include a method comprising administering a MSC/CD34+ HSPC co-cultured TCM to a subject.

In some embodiments, the MSC/CD34+ HSPC co-cultured TCM comprises substances selected from the group consisting of: cytokines, chemokines, anti-inflammatory factors, growth factors, hormones, and/or extracellular matrix components.

In some embodiments, the subject suffers from damage to a tissue, trauma to a tissue, and/or loss of a tissue.

In some embodiments, the MSC/CD34+ HSPC co-cultured TCM is administered to the damaged tissue and/or to the traumatized tissue.

In some embodiments, the method further comprises providing a graft to a subject.

In some embodiments, the MSC/CD34+ HSPC co-cultured TCM is administered after the graft.

In some embodiments, administering comprises instilling the MSC/CD34+ HSPC co-cultured TCM into the bladder of the subject.

In some embodiments, instilling comprises filling the bladder with a solution and holding the solution for a period of time.

In some embodiments, the period of time is selected from the group consisting of: 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, and 30 minutes.

In some embodiments, the method further comprises repeating the administration.

In some embodiments, the repeating is once a day, twice a day, once a week, twice a week, three times a week, four times a week, five times a week, six times a week, or seven times a week.

In some embodiments, the method further comprises continuing the administration for 1 week, 2 weeks, 3 weeks, 4 weeks, 5 weeks, 10 weeks, 20 weeks, 30 weeks, or 40 weeks.

In some embodiments, the percent vasculature of the blood vessels observed in tissue following administration is higher than the percent vasculature of the blood vessels observed in tissue without administration.

In some embodiments, the method further comprises increasing the solution volume per week to a final volume as determined by the volume at which urethral leakage occurs and continuing the administration for a period of more than one week.

In some embodiments, the MSC/CD34+ HSPC co-cultured TCM is administered in a dosage unit formulation selected from the group consisting of: conventional non-toxic pharmaceutically acceptable carriers, adjuvants, and vehicles.

In some embodiments, the tissue is selected from the group consisting of: epithelial tissue, fibrous tissue, cartilage tissue, bone tissue, blood vessels, muscle tissue, and nerve tissue.

In some embodiments, the subject suffers from bladder disease.

In some embodiments, the bladder disease is selected from the group consisting of: cystitis, interstitial cystitis, overactive bladder, bladder cancer, bladder stones, and bladder prolapse.

Embodiments of the present disclosure also include a method comprising (1) administering to the subject a first therapy comprising providing a tissue graft to a subject and, thereafter, administering to the subject a composition comprising a MSC/CD34+ HSPC co-cultured TCM and (2) administering to the subject a second therapy.

In some embodiments, the second therapy is regenerative therapy selected from the group consisting of: cell therapy, immunomodulation therapy, tissue engineering, Visco supplementation, and prolotherapy

In some embodiments, the second therapy is selected from the group consisting of: blood tests, imaging tests, physical exams, and/or iodine uptake tests.

In some embodiments, the graft is selected from the group consisting of: a seeded or an unseeded graft, one or more cells on a scaffold, and tissue.

### BRIEF DESCRIPTION OF THE DRAWINGS

FIGS. 1A-1E show urinary bladder smooth muscle regeneration, urothelium and vasculature assessment. FIG. A shows trichrome staining of bladder tissue in control and TCM animals. FIG. B shows manual measurement and quantification of urothelium width (μm). Data demonstrate TCM group had significantly greater average urothelial width than control group. FIG. C shows manual measurement and quantification of muscle:collagen. TCM group had significantly greater percent muscle content than control group. Fig. D shows manual measurement and quantification of the number and size of blood vessels in each image. TCM group demonstrated significantly greater percent vasculature compared with control group. FIG. E shows immunofluorescence co-staining of Caldesmon (green)+Smooth muscle myosin heavy chain (SMMHC) (red) and Calponin (green)+SMMHC which demonstrate greater overall protein expression in TCM regenerated tissue when compared to the control group. Immunofluorescence co-staining of vWF (green) and CD31 (red) in the third row demonstrate greater expression of markers of endothelial cell microvasculature in the TCM tissue compared to control group. p<0.05 was considered statistically significant. Data represents means±SE. 10× Scale bars represents 200 μm, 40× Scale bars represents 50 μm.

FIGS. 2A-2B show bladder βIII tubulin expression. FIG. 2A shows immunofluorescence staining of control bladder tissue for nerve-regeneration related marker, PHI tubulin. FIG. 2B shows immunofluorescence staining of treatment bladder tissue for nerve-regeneration related marker, βIII tubulin. Images depict minimal to no nerve growth in the control animals. TCM grafts demonstrated significant nerve growth and longer nerve length in all regions of the graft. 10× Scale bars represents 200 μm, 40× Scale bars represents 50 μm.

FIGS. 3A and 3B show H&E Staining of Bladder and Kidney Tissue 4-weeks Post-augmentation. At 4-weeks post-augmentation, animals were euthanized and bladders and kidneys were harvested, processed, and stained with Hematoxylin and Eosin (H&E). FIG. 3A shows images of bladder tissue (10× and 40×) demonstrating normal urothelial and smooth muscle architecture in media-instilled control animals and images of kidney tissue (10× and 40×) demonstrating normal architecture with no evidence of hydronephrosis microscopically in media-instilled control animals. FIG. 3B shows images of bladder tissue (10× and 40×) demonstrating normal urothelial and smooth muscle architecture in TCM animals. Images of kidney tissue (10× and 40×) demonstrating normal architecture with no evidence of hydronephrosis microscopically in TCM animals. 10× Scale bars represents 200 μm, 40× Scale bars represents 50 μm.

FIG. 4 Shows gross images of bladder and kidneys 4-weeks post augmentation. 4-weeks post-augmentation, animals were euthanized and bladders and kidneys were harvested intact. FIG. 4A shows images of control group animals. FIG. 4B shows images of TCM group animals. Images depict no evidence of bladder stone formation or hydronephrosis in all 9 animals. All control and treatment group animals displayed similar gross tissue appearance.

FIG. 5 shows urodynamic Studies. Urodynamic studies were performed pre- and 4-weeks post-augmentation. Intravesical pressures were directly measured and plotted as a function of time during bladder filling in both the control and TCM treatment groups.

### DETAILED DESCRIPTION

Embodiments of the present disclosure provide compositions and methods related to tissue regeneration using human mesenchymal stem cell (MSC)/CD34+ hematopoietic stem/progenitor cell (HSPC) co-cultured total conditioned media. In particular, the present disclosure provides novel compositions and methods for treating and/or preventing damage to, trauma to, and/or loss of tissue.

Section headings as used in this section and the entire disclosure herein are merely for organizational purposes and are not intended to be limiting.

### 1. DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Further, unless otherwise required by context, singular terms shall include pluralities and plural terms shall include the singular. Accordingly, in the context of the embodiments described herein, the following definitions apply.

As used herein, the singular forms “a,” “an” and “the” include plural reference unless the context clearly dictates otherwise. Thus, for example, reference to “a domain” is a reference to one or more domains and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

The terms “include(s),” “having,” “has,” “can,” “contain(s),” and variants thereof, as used herein, are intended to be open-ended transitional phrases, terms, or words that do not preclude the possibility of additional acts or structures.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “total conditioned media” refers to a culture media containing biologically active components obtained from previously cultured cells or tissues that have released into the media certain substances (e.g., cytokines, chemokines, anti-inflammatory factors, growth factors (e.g., skin cells (e.g., epidermal growth factor, EGF) nerve cells (e.g., nerve growth factor, NGF) connective tissue or mesenchymal cells (e.g., fibroblast growth factor, FGF) thrombus-forming cells that line blood vessels (e.g., platelet-derived growth factor, PDGF)), hormones (e.g., glucagon, insulin, luteinizing hormone (LH), melatonin, oxytonin, parathyroid hormone, progesterone, prolactin, testosterone, and thyroid hormone), and extracellular matrix components (e.g., elastin, fibrillin, fibulins, fibrinogen, fibronectin, laminin, tenascins and thrombospondins)) which affect certain cell functions (e.g., growth, lysis).

As used herein, the terms “administration of” and “administering” a composition (e.g., total conditioned media obtained from the co-culture of mesenchymal stem cells and CD34+ HSPCs) refers to providing a composition of the present disclosure to a subject in need of treatment (e.g., instillation treatment (e.g., bladder instillation (e.g., a procedure used to treat inflammation of the bladder wall and lining (e.g., cystitis))). The compositions of the present disclosure may be administered by generally known methods of instillation (e.g., methods of bladder instillation (e.g., filling the bladder with a solution (e.g., a buffer comprising a known or unknown concentration of the composition) and holding that solution for a period of time (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes)))) in suitable dosage unit formulations containing conventional non-toxic pharmaceutically acceptable carriers, adjuvants, and vehicles if and/or as appropriate for each route of administration.

As used herein, the terms “co-administration” and “co-administering” refer to the administration of at least two agent(s) or therapies to a subject (e.g., TCM from the co-culture of mesenchymal stem cells and CD34+ HSPCs and one or more additional therapeutics). In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the terms “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, macaque, etc.) and a human). In some embodiments, the subject may be a human or a non-human. In some embodiments, the subject is a human. The subject or patient may be undergoing various forms of treatment.

For any of the embodiments described herein, any suitable sample type may be used. The term “sample” means fluids (e.g., amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood (e.g., whole blood), blood product (e.g., plasma, serum), pleural, semen, synovial fluid, tears, thoracentesis, saliva, gargle, or urine, etc.), solids, tissues (e.g., connective tissue, epithelial tissue, muscle tissue, and nervous tissue (e.g., bladder tissues and cardiac tissues)), and gases.

As used herein, the terms “treat, “treating,” “treatment,” and variations thereof refer to the clinical intervention made in response to a disease, disorder or physiological condition manifested by a patient. The aim of treatment includes the alleviation of symptoms, slowing or stopping the progression or worsening of a disease, disorder, or condition and/or the remission of the disease, disorder, or condition. A positive response to treatment may indicate a complete response to treatment, a partial response to treatment, or a stable disease state in the subject. A treatment may be either performed in an acute or chronic way. The term also refers to reducing the severity of a disease or symptoms associated with such disease.

As used herein, the terms “prevent,” “preventing,” “prevention,” and variations thereof refers to a clinical intervention made in response to a disease, disorder or physiological condition to which a patient may be susceptible. The aim of prevention includes slowing or stopping the development of a disease, disorder, or condition. A positive response to prevention may include the subject not developing the disease, disorder or physiological condition, or the subject developing a milder version or slower progression of the disease, disorder or physiological condition. Additionally, preventing may reduce the likelihood that a subject within a population of susceptible individuals will develop a disease, disorder or physiological condition, even though any particular individual may still develop the disease, disorder or physiological condition. A prevention may be either performed in an acute or chronic way. Preventing also refers to preventing the recurrence of a disease or of one or more symptoms associated with such disease.

As used herein, the term “tissue regeneration” means regeneration of epithelial tissue, regeneration of fibrous tissue, regeneration of cartilage tissue, regeneration of bone tissue, regeneration of blood vessels, regeneration of muscle tissue, and regeneration of nerve tissue. As used herein, the term “regeneration” means the action or process of regenerating or being regenerated, in particular the structure and formation of new tissue.

As used herein, the term “secretome,” or “cell secretome,” and variations thereof refer to the paracrine and autocrine cell signaling mechanism which regulate many physiological processes.

### 2. COMPOSITIONS

Embodiments of the present disclosure include compositions comprising a culture media containing biologically active components obtained from (i.e., derived from) previously cultured mesenchymal stem cells (MSC) and previously cultured CD34+ hematopoietic stem/progenitor cells (HSPC).

In some embodiments, culture media is a total conditioned media (TCM). In some embodiments, the culture media is a TCM derived from co-culturing MSCs and CD34+ HSPCs (e.g., a MSC/CD34+ HSPC co-cultured TCM). In some embodiments, the culture media is a TCM derived from a single cell culture (e.g., a MSC or a CD34+ HSPC). In some embodiments, the culture media is a TCM derived from the combination of separate single cell cultures (e.g., a MSC and a CD34+ HSPC).

In some embodiments, the culture media or the TCM comprises biologically active components obtained from previously cultured cells or tissues that have released into the media certain substances (e.g., cytokines, chemokines, anti-inflammatory factors, growth factors (e.g., skin cells (e.g., epidermal growth factor, EGF) nerve cells (e.g., nerve growth factor, NGF) connective tissue or mesenchymal cells (e.g., fibroblast growth factor, FGF) thrombus-forming cells that line blood vessels (e.g., platelet-derived growth factor, PDGF)), hormones (e.g., glucagon, insulin, luteinizing hormone (LH), melatonin, oxytonin, parathyroid hormone, progesterone, prolactin, testosterone, and thyroid hormone), and extracellular matrix components (e.g., elastin, fibrillin, fibulins, fibrinogen, fibronectin, laminin, tenascins and thrombospondins)) which affect certain cell functions (e.g., growth, lysis), small and large molecules, DNA, RNA, intracellular vesicles.

In some embodiments, the culture media or the TCM comprises pro-inflammatory cytokines, such as, IL-1α (Interleukin-1 alpha), IL-1β (Interleukin-1 beta), IL-6 (Interleukin-6), TNF-α (Tumor Necrosis Factor-alpha), IFN-γ (Interferon-gamma), IL-17A (Interleukin-17A), IL-18 (Interleukin-18), GM-CSF (Granulocyte-macrophage colony-stimulating factor), etc.

In some embodiments, the culture media or the TCM comprises growth and hematopoietic factors, such as, VEGF (Vascular Endothelial Growth Factor), EGF (Epidermal Growth Factor), FGF2 (Basic Fibroblast Growth Factor), PDGF (Platelet-Derived Growth Factor), SCF (Stem Cell Factor, aka KIT ligand), G-CSF (Granulocyte colony-stimulating factor), M-CSF (Macrophage colony-stimulating factor), etc.

In some embodiments, the culture media or the TCM comprises anti-inflammatory or immunoregulatory cytokines, such as, IL-10 (Interleukin-10), TGF-β (Transforming Growth Factor-beta), IL-4 (Interleukin-4), IL-13 (Interleukin-13), etc.

In some embodiments, the culture media or the TCM comprises chemokines (chemoattractant cytokines), such as, CXCL8 (also known as IL-8), CCL2 (MCP-1, Monocyte Chemoattractant Protein-1), CCL5 (RANTES), CXCL10 (IP-10, Interferon gamma-induced protein 10), CXCL12 (SDF-1, Stromal cell-derived factor 1), etc.

In some embodiments, the culture media or the TCM comprises extracellular matrix (ECM) components, such as, collagens, elastin, fibronectin, laminins, tenascin, vitronectin, proteoglycans and glycosaminoglycans (e.g., decorin, biglycan, perlecan, aggrecan, versican, syndecans & glypicans, common GAGs (e.g., hyaluronic acid, chondroitin sulfate, heparan sulfate, dermatan sulfate, keratan sulfate, etc.), etc.), ECM-modifying enzymes, thrombospondins, osteopontin, SPARC (secreted protein acidic and rich in cysteine), fibrillin, etc.

In some embodiments, the biologically active components are obtained from (i.e., derived from) previously cultured mesenchymal stem cells (MSC) and previously cultured CD34+ hematopoietic stem/progenitor cells (HSPC) (e.g., a MSC/CD34+ HSPC co-cultured TCM). In some embodiments, the MSCs and HSPCs are human cells.

In some embodiments, the previously cultured MSCs are obtained from (i.e., derived from) human bone marrow. In some embodiments, the previously cultured MSCs are obtained from (i.e., derived from) a non-bone marrow source (e.g., adipose tissue). In some embodiments, the previously cultured MSCs are thawed and seeded at a density of approximately 4,000 viable cells/cm² (100,000 cells/25 cm² flask) in media designed for deriving, expanding, and/or cryopreserving MSCs (e.g., MesenCult-ACF Plus Medium). In some embodiments, the previously cultured MSCs are thawed and seeded for 1 hour, 6 hours, 12 hours, 24 hours, 36 hours, 72 hours, or 84 hours.

In some embodiments, the previously cultured CD34+ HSPCs are obtained from (i.e., derived from) human bone marrow. In some embodiments, the previously cultured CD34+ HSPCs are thawed and co-seeded with the MSCs at a concentration of approximately 4,000 viable cells/cm².

In some embodiments, the previously cultured MSCs and CD34+ HSPCs are cultured in a 1:1 mixture of media designed for the culture and expansion of hematopoietic cells isolated from human, non-human primate, and/or mouse blood and/or bone marrow to media specifically designed for deriving, expanding, and/or cryopreserving MSCs (e.g., MesenCult-ACF Plus Medium) (e.g., a 1:1 mixture of StemSpan™-XF: MesenCult™-ACF Plus Medium).

In some embodiments, prior to administration to a subject (e.g., prior to bladder instillation), the MSCs and CD34+ HSPCs (e.g., the co-seeded cell populations in each culture flask) in the culture media or TCM are pelleted by centrifugation at a speed of approximately 1000 relative centrifugal force (RCF) for approximately 5 minutes (although other protocols are within the scope herein). In some embodiments, an aliquot of the resulting (e.g., fresh) supernatant of the culture media or TCM is collected. In some embodiments, the collected culture media or TCM is used in administration to a subject (e.g., used in a bladder instillation).

In some embodiments, the pelleted cells are resuspended and plated into the same culture flask using two (2) mL of a 1:1 mixture of media designed for the culture and expansion of hematopoietic cells isolated from human, non-human primate, and/or mouse blood and/or bone marrow to media specifically designed for deriving, expanding, and/or cryopreserving MSCs (e.g., MesenCult-ACF Plus Medium) (e.g., a fresh 1:1 mixture of StemSpan™-XF: MesenCult™-ACF Plus Medium) (although other protocols are within the scope herein).

In some embodiments, the composition furthers comprises a scaffold (e.g., a fibrous, porous, and hydrogel scaffolds). In some embodiments, the scaffold is configured to provide localized, sustained, or controlled release. Such scaffolds may be biodegradable or non-biodegradable and may be fabricated from natural, synthetic, or semi-synthetic materials. Suitable scaffold materials include, without limitation, collagen, gelatin, fibrin, hyaluronic acid, chitosan, alginate, polylactic acid (PLA), polyglycolic acid (PGA), poly(lactic-co-glycolic acid) (PLGA), polycaprolactone (PCL), polyethylene glycol (PEG)-based hydrogels, and other biocompatible polymers. The scaffold may be in the form of a porous matrix, sponge, gel, hydrogel, mesh, film, microsphere, nanofiber mat, or three-dimensional printed structure. In some embodiments, the scaffold mimics the extracellular matrix and supports cellular infiltration, angiogenesis, and tissue regeneration while gradually releasing therapeutic agents. The release kinetics may be modulated by the scaffold's composition, porosity, crosslinking density, degradation rate, and incorporation method (e.g., physical entrapment, covalent conjugation, or encapsulation in microparticles embedded within the scaffold). In some embodiments, the composition furthers comprises a bladder acellular matrix (BAM) scaffold. In some embodiments, the composition furthers comprises a small intestinal submucosa (SIS) scaffold. In some embodiments, the composition furthers comprises a synthetic biodegradable polyester (e.g., polycaprolactone (PCL), polyglycolide (PGA), polylactide (PLA) and their copolyesters poly(lactide-co-glycolide) (PLGA)) scaffold.

In some embodiments, the subject is provided with a graft (e.g., a seeded or an unseeded graft). In some embodiments, the graft is a cold-preserved graft. In some embodiments, the graft is one or more cells (e.g., MSCs and/or CD34+ HSPCs) on a scaffold. In some embodiments, the graft is tissue (e.g., bladder tissue, liver tissue, pancreas tissue, kidney tissue, adrenal gland tissue, lung tissue, and/or cardiovascular tissue).

### 3. METHODS

Embodiments of the present disclosure also include methods comprising cell-free tissue regeneration approaches.

In some embodiments, the methods comprise administering a composition to a subject, wherein the composition comprises a culture media containing biologically active components obtained from (i.e., derived from) previously cultured mesenchymal stem cells (MSC) and previously cultured CD34+ hematopoietic stem/progenitor cells (HSPC) (e.g., a MSC/CD34+ HSPC co-cultured TCM).

In some embodiments, the subject is in need of treating and/or preventing damage to, trauma to, and/or loss of tissue. The tissue at risk may include, without limitation, epithelial tissue, connective tissue, muscle tissue, nervous tissue, vascular tissue, hematopoietic and lymphoid tissue, and organ-specific parenchymal tissue.

Epithelial tissue encompasses the outer skin layers and the linings of internal organs, cavities, and vessels. Damage to epithelial tissue can result from physical trauma such as abrasions or surgical incisions, thermal or chemical burns, infections (including bacterial, viral, or fungal), autoimmune disorders like pemphigus vulgaris, and chronic non-healing wounds such as diabetic ulcers and pressure sores.

Connective tissue, which includes bone, cartilage, ligaments, tendons, and adipose tissue, may be damaged by fractures, joint dislocations, ligament sprains, tendon ruptures, or degenerative conditions such as osteoarthritis. It may also be compromised during surgical procedures involving resection or repair. Muscle tissue—including skeletal, cardiac, and smooth muscle—can be injured by mechanical trauma, overuse, or contusions, as well as by inherited or acquired myopathies, ischemic injury (as seen in myocardial infarction), neurodegenerative disorders, and toxin-induced damage, such as that caused by certain medications.

Nervous tissue, comprising both the central and peripheral nervous systems, may be subject to damage through traumatic brain or spinal cord injuries, stroke, neuropathies like diabetic neuropathy, inflammatory conditions such as multiple sclerosis, and iatrogenic injury during surgery. Vascular tissue, including arteries, veins, and capillaries, may be damaged due to atherosclerosis, inflammation (vasculitis), radiation exposure, surgical manipulation, or microvascular complications of diabetes.

Hematopoietic and lymphoid tissues, such as bone marrow, lymph nodes, spleen, and thymus, are vulnerable to injury from chemotherapy, radiation, autoimmune attack (e.g., aplastic anemia), viral infections (e.g., HIV or EBV), or malignancies of hematopoietic origin. Additionally, organ-specific parenchymal tissue, such as that of the liver, kidneys, lungs, pancreas, or heart, may sustain damage from ischemia or infarction, drug toxicity (e.g., acetaminophen-induced liver failure), inflammation (e.g., hepatitis or pancreatitis), transplant-related complications, or chronic degenerative conditions like cirrhosis or chronic obstructive pulmonary disease.

In various embodiments, the subject may be at risk of tissue damage due to acute trauma, chronic disease, therapeutic interventions (including surgery, radiation, or immunosuppression), or pathological processes such as infection or autoimmune dysregulation. The present disclosure contemplates methods, compositions, and devices useful in treating, mitigating, or preventing such tissue damage, promoting tissue repair and regeneration, or otherwise preserving the structure and function of the affected tissue.

In some embodiments, the subject is in need of treating and/or preventing damage to, trauma to, and/or loss of cardiac tissue or lung tissue. In some embodiments, the subject is in need of treating and/or preventing damage to, and/or trauma to joints. In some embodiments, the subject suffers from damage to, trauma to, and/or loss of tissue. In some embodiments, the tissue is epithelial tissue, fibrous tissue, cartilage tissue, bone tissue, blood vessels, muscle tissue, and/or nerve tissue. In some embodiments, the tissue is cardiac tissue. In some embodiments, the tissue is lung tissue.

In some embodiments, the subject is provided with a graft (e.g., a seeded or an unseeded graft). In some embodiments, the graft is a cold-preserved graft.

In some embodiments, administration is by generally known methods of actuation (e.g., use of an actuator).

In some embodiments, administration is by generally known methods of timed release from a scaffold (e.g., a scaffold as described herein).

In some embodiments, administration is by generally known methods of instillation (e.g., methods of bladder instillation (e.g., filling the bladder with a solution (e.g., a buffer comprising a known or unknown concentration of the composition) and holding that solution for a period of time (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes)))). In some embodiments, the methods of instillation are repeated (e.g., once a day, twice a day, once a week, twice a week, three times a week, four times a week, five times a week, six times a week, seven times a week). In some embodiments, the composition is changed 2 hours, 4 hours, 6 hours, 8 hours, 10 hours, 12 hours, 14 hours, 16 hours. 18 hours, 20 hours, 22 hours. 24 hours. 26 hours, 28 hours, or 30 hours prior to each repetition of instillation. In some embodiments, the repetitions of instillation are administered for a period of time (e.g., 1 week, 2 weeks, 3 weeks, 4 weeks, 5 weeks, 10 weeks, 20 weeks, 30 weeks, 40 weeks). In some embodiments, the methods of instillation comprise increasing volumes of TCM or media as determined by the volume at which urethral leakage occurs. In some embodiments, the increase in volume of TCM or media is 20 uL increase per week, 30 uL increase per week, 40 uL increase per week, 50 uL increase per week, 60 uL increase per week, 70 uL increase per week, 80 uL increase per week, 90 uL increase per week, 100 uL increase per week, 120 uL increase per week, 130 uL increase per week, 140 uL increase per week, or 150 uL increase per week. In some embodiments, the increase in volume of TCM or media is 200 uL increase per week, 400 uL increase per week, 800 uL increase per week). In some embodiments, the methods of instillation comprise reaching a final instillation capacity of 100 uL, 200 uL, 300 uL, 400 uL, 500 uL, 600 uL, 700 uL, 800 uL, 900 uL, 1000 uL.

In some embodiments, following administration, the percent vasculature of the blood vessels observed is greater than about two percent (e.g., 1.50%, 1.75%, 2.00%, 2.50%, 2.75%, 3.00%, 4.00%, 5.00%).

In some embodiments, the composition is administered in suitable dosage unit formulations containing conventional non-toxic pharmaceutically acceptable carriers, adjuvants, and vehicles if and/or as appropriate for each route of administration.

In some embodiments, the composition is administered in one or more suitable dosage units (e.g., 1, 2, 3, 4, 5, 6, 7, 8, 9, 10).

In some embodiments, the subject suffers from bladder disease. In some embodiments, the bladder disease is cystitis, interstitial cystitis, overactive bladder, bladder cancer, bladder stones, and/or bladder prolapse, neurogenic bladder.

In some embodiments, the subject suffers from liver disease. In some embodiments, the liver disease is hepatitis (e.g., hepatitis A, hepatitis B, hepatitis C, autoimmune hepatitis) cirrhosis, alcohol-associated liver disease (e.g., liver diseases caused by too much alcohol consumption (e.g., alcohol-associated fatty liver, alcohol-associated hepatitis, alcohol-associated cirrhosis), nonalcoholic fatty liver disease (e.g., nonalcoholic steatohepatitis), primary biliary cholangitis, primary sclerosing cholangitis, liver cancer, bile duct cancer, and/or liver cell adenoma.

In some embodiments, the subject suffers from pancreatic disease (e.g., redness and swelling (e.g., inflammation) of the pancreas (e.g., pancreatitis (e.g., acute or chronic pancreatitis)).

In some embodiments, the subject suffers from thyroid disease. In some embodiments, the thyroid disease is hyperthyroidism (e.g., Graves' disease (e.g., production of too much thyroid hormone)), toxic adenomas (e.g., nodules that form in the thyroid gland and upset the subject's chemical balance by making thyroid hormones), subacute thyroiditis (e.g., inflammation of the thyroid that causes the gland to leak excess hormones), pituitary gland malfunctions or cancerous growths in the thyroid gland). In some embodiments, the thyroid disease is hypothyroidism (e.g., primary hypothyroidism (e.g., when the thyroid gland itself is not able to produce adequate amounts of thyroid hormone (e.g., Hashimoto's), central hypothyroidism (e.g., when the thyroid gland is normal and the pathology is related to the pituitary gland or hypothalamus), and tertiary hypothyroidism).

In some embodiments, the subject suffers from kidney disease (e.g., acute and chronic kidney disease).

In some embodiments, the subject suffers from disease of the adrenal glands (e.g., Addison's disease and Cushing's syndrome).

In some embodiments, the subject suffers from lung disease (e.g., a disease that affects the ability of the subject to breathe and/or a disease that affects the ability of the subject's lungs to work and/or function). In some embodiments, the lung disease results from a bacterial, a viral, or a fungal infection (e.g., the lung disease is pneumonia, tuberculosis, and/or influenza). In some embodiments, the lung disease results from environmental factors (e.g., the lung disease is asthma, mesothelioma, and/or lung cancer). In some embodiments, the lung disease results uncontrolled cell division in the lungs (e.g., the lung disease is cancer). In some embodiments, the lung disease comprises collapse of the lung (e.g., pneumothorax and/or atelectasis), swelling and inflammation in the bronchial tubes (e.g., bronchitis), abnormal fluid buildup in the lungs (e.g., pulmonary edema), and damage to the alveoli (e.g., emphysema). In some embodiments, the lung disease is a chronic lower respiratory disease (e.g., chronic obstructive pulmonary disease (COPD), emphysema, and/or chronic bronchitis).

In some embodiments, the subject suffers from cardiovascular disease. In some embodiments, the cardiovascular disease is coronary heart disease (e.g., a disease of the blood vessels supplying the heart muscle), cerebrovascular disease (e.g., a disease of the blood vessels supplying the brain), peripheral arterial disease (e.g., a disease of blood vessels supplying the arms and legs), rheumatic heart disease (e.g., damage to the heart muscle and heart valves from rheumatic fever, caused by streptococcal bacteria), congenital heart disease (e.g., birth defects that affect the normal development and functioning of the heart caused by malformations of the heart structure from birth), and/or deep vein thrombosis and pulmonary embolism (e.g., blood clots in the leg veins, which can dislodge and move to the heart and lungs).

In some embodiments, the treatment further comprises administering a second regenerative therapy. In some embodiments, the second regenerative therapy is cell therapy (e.g., injection of stem cells or progenitor cells (e.g., stem cell transplants)), immunomodulation therapy (e.g., regeneration by biologically active molecules administered alone or as secretions by infused cells platelet-rich plasma (e.g., injections of the subject's own blood)), tissue engineering (e.g., transplantation of laboratory grown organs and tissues), Viscosupplementation (e.g., gel-like substances, hyaluronates (e.g., Hyalganm Supartz, Synvisc, Euflexxa, and/or Orthovisc), that mimic the properties of naturally occurring joint fluid), and/or prolotherapy (e.g., injections of an irritant).

In some embodiments, the subject suffers from joint disease (e.g., a disease that affects the health and function of joints, bones, tendons, ligaments, cartilage, and/or muscles). In some embodiments, the joint disease is osteoarthritis (e.g., a long-term condition that causes pain and limits joint movement (e.g., joint degeneration or degenerative joint disease)). In some embodiments, the joint disease is rheumatoid arthritis (e.g., an autoimmune condition that affects joints and causes pain, swelling, and inflammation). In some embodiments, the joint disease is gout (e.g., a type of arthritis caused by a buildup of uric acid in the joints). In some embodiments, the joint disease is bursitis (e.g., a condition where the bursae become inflamed). In some embodiments, the joint disease necessitates joint reconstruction (e.g., anterior cruciate ligament reconstruction).

In some embodiments, the treatment further comprises blood tests, imaging tests, physical exams, and/or iodine uptake tests.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein.

Having now described the present disclosure in detail, the same will be more clearly understood by reference to the following examples, which are merely intended only to illustrate some aspects and embodiments of the disclosure, and should not be viewed as limiting to the scope of the disclosure.

The disclosures of all journal references, U.S. patents, and publications referred to herein are hereby incorporated by reference in their entireties.

The present disclosure has multiple aspects, illustrated by the following non-limiting examples.

### Example 1

Disclosed herein is tri-layer tissue regeneration approach offers a cell-free alternative to promotion of bladder regeneration in patients augmented with an elastic synthetic scaffold (PRS), thereby obviating the need for cell acquisition or long-term cell culture.

Bladder tissue regenerative capacity of human MSC/CD34+ HSPC co-cultured total conditioned media (TCM) is compared to media alone in an immune-competent rat bladder augmentation model where rats underwent partial cystectomy and were augmented with an unseeded PRS graft. Augmented bladders were either instilled with media (control, n=4) or TCM (n=5) twice a week for 4 weeks. Regenerated tissue was analyzed for smooth muscle, urothelium, vascular, and peripheral nerve regrowth. Urodynamic (UDS) measures were performed pre- and 4 weeks post-augmentation. The results demonstrate that TCM-instilled grafts had significantly greater muscle content, larger average urothelial widths, higher percent vascularization, and more robust neural infiltration post-augmentation. UDS demonstrates greater percent bladder recovery in the TCM group, indicating functional improvement in bladder storage capacity. This study is the first to propose and demonstrate the use of cell-free TCM as an alternative to traditional cell-seeded scaffolds to promote bladder tissue regeneration.

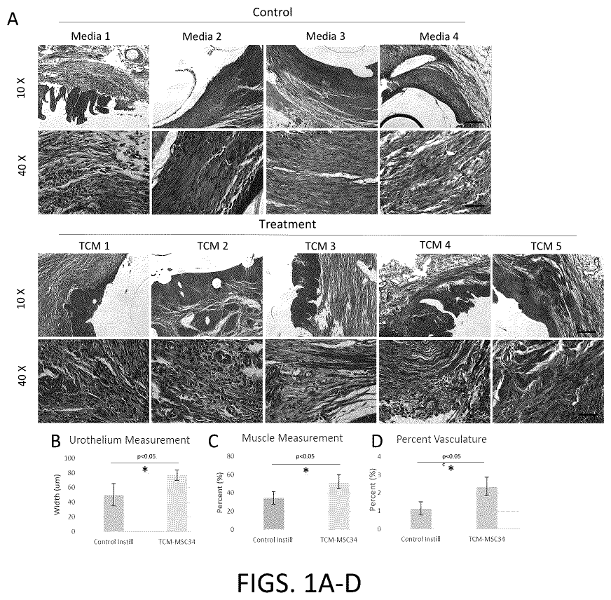

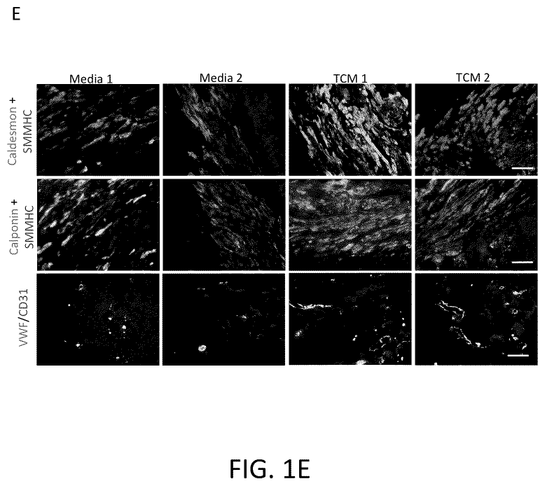

Blood vessel quantification and microvasculature staining. Blood vessel formation is a crucial component of bladder tissue regeneration, as this ensures adequate oxygenation and nutrient delivery to the graft. Blood vessel formation in all experimental and control groups following tissue processing was quantified (FIG. 1A). Mean vessel number in regenerated tissue was similar between the TCM-instilled and control animal groups (82.5±7.6 vessels/mm2 vs 75.9±3.6 vessels/mm², p>0.05, n.s.). However, there was a substantial difference in the mean blood vessel size. This was represented as percent vasculature observed in each imaged section of regenerated tissue (FIG. 1D). The TCM group demonstrated 2.40%±0.51 vascularization while the control group showed 1.14%±0.37 vascularization (p<0.05). This indicated that, while the number of vessel growth in the regenerated regions are similar if bladders are instilled with TCM or media, the size of the vessels are much larger and prominent in tissues instilled with TCM. As an adjunct, immunofluorescence co-staining of vWF and CD31 (FIG. 1E), which are endothelial cell markers seen in vascular beds in human tissue, demonstrated higher global expression in the TCM group compared to the media control. Taken together, these findings demonstrate overall greater revascularization in animals instilled with TCM.

Muscle and urothelial quantification. Masson's trichrome staining was used to assess muscle to collagen ratios in all regenerated tissue (FIG. 1A). Previous data demonstrated that unseeded grafts of athymic nude rats contained 20% muscle 4 weeks post-augmentation. In the present study, TCM-instilled animals displayed 52.2%±7.7 muscle/collagen content while control animals had 34.3%±6.9 (p<0.05) (FIG. 1C). Normal muscle/collagen ratios in the rat are approximately 1:1. Immunofluorescence co-staining of Caldesmon+Smooth muscle myosin heavy chain (SMMHC) and Calponin+SMMHC demonstrated greater overall expression of smooth muscle-related proteins in TCM regenerated tissue when compared to graft tissue from the control group (FIG. 1E). Urothelium width (μm) was measured using Masson's trichrome-stained images. Urothelium width measurements were made from the basal layer of the urothelium to its apical layer from multiple aspects of the regenerated bladder tissue. Rats have a normal urothelium width that ranges from 50-70 μm. The data demonstrated that TCM animals maintained a mean width of 76.6±7.4 μm while control animals had a mean width of 50.1±15.1 μm width (p<0.05) (FIG. 1B). This confirmed appropriate urothelial cell recruitment to regions of tissue regeneration in the presence of cellular-derived TCM.

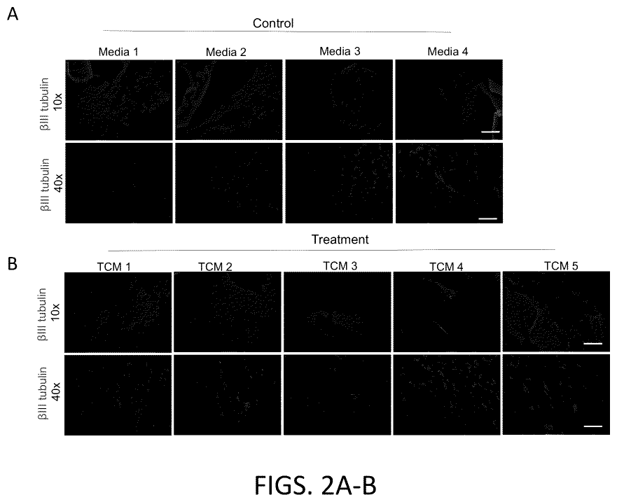

Peripheral Nerve Regeneration. Explanted native and regenerated bladder tissue areas were stained with neuronal-specific antibody βIII tubulin to determine the effect of TCM instillation on bladder peripheral nerve regeneration (FIG. 2). All five TCM-instilled animals revealed nerve infiltration on all sides of the graft 4 weeks post-augmentation, while only 2 of 4 control animals demonstrated minimal neuronal growth on one side of each graft (Table 1). The maximum average nerve regeneration distance in the TCM treated group (n=5 demonstrating nerve growth) was 1000.5±238.7 μm vs 460.8±71.8 μm in the control group (n=2 demonstrating nerve growth) (p<0.001). The mean nerve length in the TCM group was 41.2±7.0 μm vs 25.2±8.7 μm in the control group (p<0.05). These findings clearly demonstrate that TCM reproducibly promotes neural growth and infiltration in regenerating bladder tissue, which has the potential to allow for complete innervation of the graft.

TABLE 1
Quantification of peripheral nerve regeneration was performed including
percent of total animals in each group with positive staining, average
nerve length, and maximum nerve regeneration. p < 0.05 was considered
statistically significant. Data represents means ± SE.

| | | Average | |
|---|---|---|---|
| | Percent of Animals | Nerve | Maximum Nerve |
| | with βIII Tubulin | Length | Regeneration |
| | Staining | [μm] | Distance [μm] |
| Media Control | 50% (2/4) | 25.16 ± 8.72 | 460.80 ± 71.84 |
| TCM Treatment | 100% (5/5) | 41.3 ± 7.03 | 1000.49 ± 238.67 |

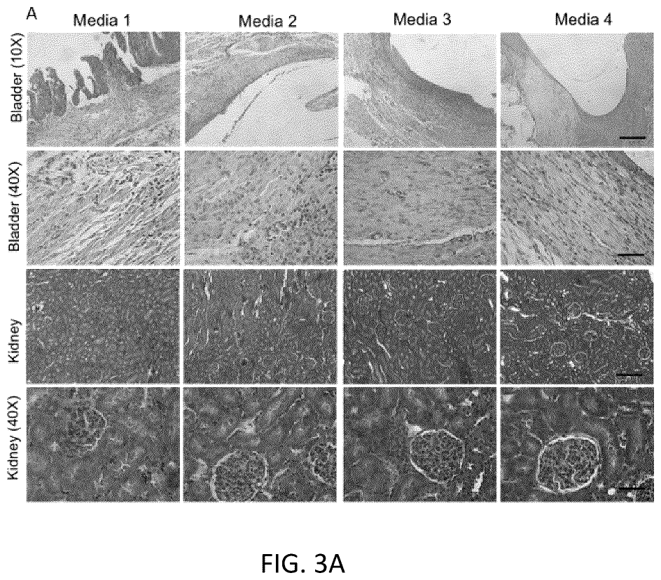

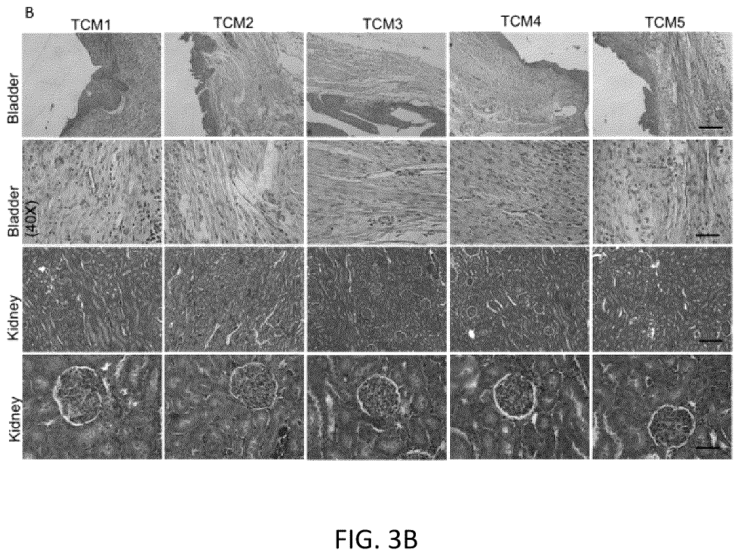

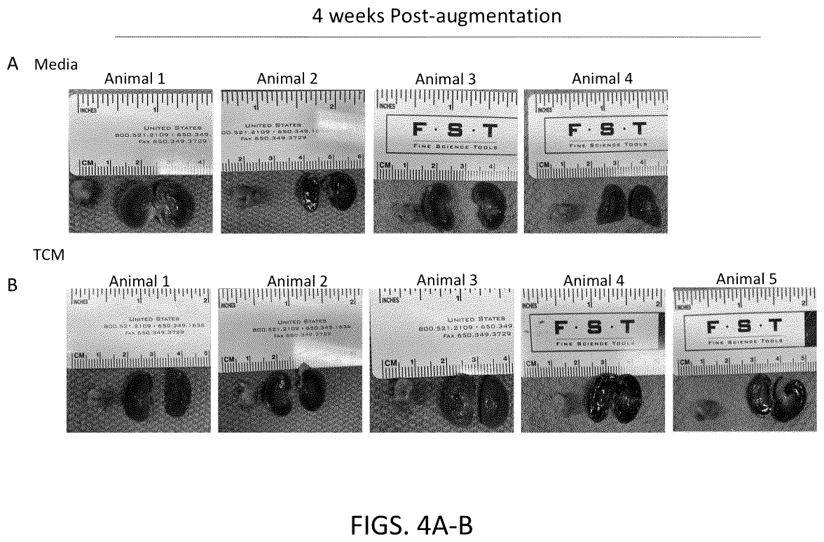

Bladder and kidney evaluation following bladder augmentation. No bladder or kidney stones were observed on gross inspection of all nine (9) animals (FIG. 4). Kidney morphology appeared normal at the gross and microscopic levels following Hematoxylin and Eosin (H&E) staining (FIG. 3A and FIG. 3B). No hydronephrosis or ureteral dilation was noted in all animals 4 weeks post-augmentation (FIG. 4).

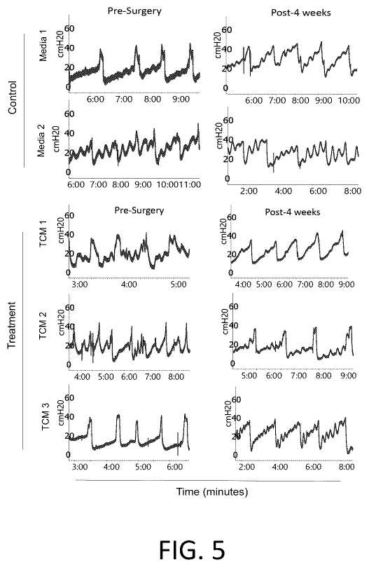

Urodynamic studies. Urodynamic studies were performed to assess bladder function pre- and post-augmentation for all groups (FIG. 5). Prior work from our group has demonstrated a rat bladder compliance (the percentage of bladder volume filled at less than 20 cm H₂O) greater than 0.50 to be physiologically normal. In the present study at 4 weeks post-augmentation, the TCM group had a mean compliance of 0.71±0.03 compared to 0.51±0.10 in the control group (p<0.005). Post-augmentation mean bladder capacity was 972.0±51.6 μl in the TCM group and 867.5±62.4 μl in the control (p<0.05). When compared to pre-augmentation capacities, the calculated mean percent bladder recovery (pre-augment capacity:post-augment capacity ratio*100) was 107.9±4.6% in the TCM group compared to 91.2±3.8% in the control group (p<0.05). Pre-augment detrusor overactivity was noted in 2 animals prior to any surgical intervention or instillation, and therefore the significance of this finding is unknown.

Experimental Methods. PRS [poly(1,8-octamethylene-citrate-co-octanol]scaffold synthesis. In order to synthesize poly (1,8-octamethylene-citrate-co-octanol) (PRS), octanol, 1,8-octanediol, and citric acid (Sigma Aldrich, St. Louis, MO) were added to a flask in a 0.2:0.8:1 molar ratio and then melted in a silicon oil bath at 165° C. with nitrogen gas flow. The mixture was stirred for about 15 min. Once the solution was melted, the flask was transferred to another oil bath at 140° C. and subjected to 3 hrs under nitrogen gas flow forming a pre-polymer. This was dissolved in ethanol and purified by precipitation in Milli-Q water with 20% ethanol, with two additional purification steps in Milli-Q water and final precipitation collection, which was frozen at −80° C. for 12-16 hrs. The pre-polymer was then lyophilized for 2-3 days until clear and dissolved in 40% ethanol w/v. PRS was characterized using proton nuclear magnetic resonance (1H-NMR, X500, Bruker) and mass spectrometry (Amazon-SL, Bruker).

Cell Culture. Human bone marrow MSCs (StemCell™ Technologies, Vancouver, BC) were thawed and seeded at a density of 4,000 viable cells/cm² (100,000 cells/25 cm² flask) in MesenCult-ACF Plus Medium (StemCell Technologies) for 72 hours. Human bone marrow CD34+ HSPCs (StemCell Technologies) were then thawed and co-seeded with the MSCs at a concentration of 4.000 viable cells/cm². The cells were cultured in a 1:1 mixture of StemSpan™-XF: MesenCult™-ACF Plus Medium (Stem Cell Technologies). Cell viability was determined by NucBlue live cell stain (Thermo Fisher Scientific, Waltham, MA) prior to seeding. The co-cultured cell media was changed 24 hr prior to each animal bladder instillation. Immediately preceding each bladder instillation, the co-seeded cell populations in each flask were pelleted by centrifugation at a speed of 1000 relative centrifugal force (RCF) for 5 minutes and fresh supernatant of total conditioned media (TCM) was collected in preparation for bladder instillations. The pelleted cells were resuspended and plated into the same culture flask in 2 mL of fresh 1:1 StemSpan™-XF: MesenCult™-ACF Plus Medium. A 1:1 mixture of StemSpan™-XF: MesenCult™-ACF Plus Medium was prepared for control group bladder instillations.

In vivo bladder augmentation studies. Sprague Dawley rats (females weighing ˜300 g; 9-10 weeks of age; Charles River Laboratories, Wilmington, MA) underwent bladder augmentation as previously described by our laboratory.^{[6]} Rats were anesthetized with inhalation of 2% isoflurane. A 1.0 cm midline incision was created with abdominal fascia and abdominal wall musculature exposed with subsequent identification of the urinary bladder. A 70% supratrigonal anterior-to-posterior cystectomy was performed and the defect was augmented with an unseeded PRS scaffold in all rats. In this study, female rats were used in order to decrease risk for urethral irritation and edema during catheterization for each bladder instillation (n=9 total animals utilized). The bladder was closed with 7-0 polyglactin suture in a watertight manner and subsequently sutured with surrounding omentum. Rats were catheterized with a 20 gauge angiocatheter and 100 uL of either co-seeded cell TCM (n=5) or 1:1 media (n=4) was instilled into the bladder. No significant leakage was noted prior to closure of the abdominal wall with 4-0 chromic running suture. The skin was then re-approximated with 9 mm autoclips. Bladder instillations were performed under anesthesia twice a week for the duration of the 4-week study period. Each instillation consisted of increasing volumes (50-100 uL increase per week) of TCM or media determined by volume at which urethral leakage was noted, reaching a final instillation capacity of 400-500 uL. All animal studies guidelines were set forth and approved by the Northwestern University Institutional Animal Care and Use Committee (IACUC).

Tissue specimen processing/staining. Whole bladders and kidneys were removed from augmented animals following euthanasia 4-weeks post-augmentation. Specimens were fixed in a 10% buffered formalin phosphate and dehydrated with exchanges of graduated ethanol. The samples were then embedded in paraffin and sectioned onto glass slides at a 5 μm thickness which were subsequently deparaffinized with xylenes, graduated ethanol washes, and deionized water. This was followed by an established staining protocol for Masson's trichrome and independent H&E staining. The specimens were consecutively deparaffinized with xylenes, dehydrated with ethanol changes and re-hydrated with deionized water. After these steps, slides were stained with H&E, followed by washes with gradient ethanol solutions. Following air-drying, a coverslip was placed over the specimen sample and secured with Permaslip (Alban Scientific Inc.). A total of 9 bladders underwent tissue analysis from all experimental groups: control (n=4) and TCM (n=5).

Blood vessel quantification in areas of bladder tissue regeneration. Sample images stained with Masson's trichrome were digitized and further characterized using a Nikon Eclipse 50i Microscope (Nikon Inc., Melville. NY). The images were opened with Adobe Photoshop CS3 (Adobe Systems Inc.). The pen tool in Adobe was utilized to quantify vessel numbers based upon n=10 images per graft in areas of regeneration. Individual vessels were manually selected and the image histogram tool was used to acquire pixel density for each vessel. Data is represented as mean number of vessels/mm² (means±SE).

Bladder tissue muscle quantification. In order to quantify muscle and collagen expression, the digitized images of all Masson's trichrome stained samples (1600 pixels-1200 pixels, bit depth 24) were opened with Adobe Photoshop CS3. A two-fold elevation of magenta levels enhanced the contrast of red pixels from blue pixels. This was followed by a two-fold depression of cyan levels in the red and magenta spectra. This was enhanced by a two-fold elevation of cyan levels and then a two-fold depression of magenta levels in the cyan and blue spectra. The selection color range tool was used to digitally select the red or blue pixels. The image histogram tool was used to quantify the selected pixels. The muscle to collagen ratio was calculated from these values, as previously described. Any images containing red blood cells, debris, urothelial cells and PRS scaffold were edited to remove these structures and thereby enhance visualization of muscle content. Data was based upon ten images per animal for each group at the 4-week time-point.

Immunofluorescent characterization of augmented tissues. Following the aforementioned deparaffinization process, tissue samples were also subjected to immunofluorescent staining. Slides underwent antigen retrieval, which consisted of 15 min of boiling in citrate buffer (0.01 m citrate solution, pH 6.0 with 0.05% Tween-20) and subsequent cooling at room temperature. The slides were blocked for 15 min in bovine serum albumin (BSA, 5 mg/ml) and then incubated at room temperature with the primary antibody (against βIII tubulin, smooth muscle myosin heavy chain (SMMHC), calponin, von Willebrand factor (vWF) and CD31. After washing with DPBS, slides were incubated for 30 min with either an Alexa Red 555 or FITC conjugated secondary antibody (Molecular Probes, Carlsbad, CA), rinsed with DPBS. Slides were mounted with Vectashield (Vector Laboratories, Burlingame, CA). Primary antibodies were diluted to working concentrations of 1 μg/mL while secondary antibodies were diluted to concentrations of 1-10 μg/ml. All samples were also stained with 4′,6-diamidino-2-phenylindole (DAPI) to visualize cellular nuclei.

Kidney and bladder evaluation. The presence or absence of kidney hydronephrosis was noted visually immediately following euthanasia. Kidney and bladder cross sections were stained with H&E and digitally imaged as previously described. Gross and microscopic evaluation of the kidney and bladder cross sections were performed.

Urodynamic studies and bladder capacity evaluation. Urodynamic studies (UDS) and bladder capacity measurements were performed prior to bladder augmentation and immediately prior to euthanization. Bladder capacity was measured prior to UDS. Bladders were emptied by manual decompression and then catheterized using 20 gauge angiocatheters (Becton Dickinson, Franklin Lakes, NJ) per urethra. Saline was added at a rate of 150 μl/min until urethral leakage was observed. Sprague Dawley rats were anesthetized as described above, and a lower abdominal incision was made to expose the bladders. A 20 gauge cannula was inserted into the bladder dome. This was connected to the Pump 11 Elite Syringe Pump (Harvard Apparatus. Holliston, MA) and to a physiological pressure transducer (SP844, MEMSCAP), which was then connected to a bridge amplifier (Model FE221; AD Instruments, Colorado Springs, CO), which plotted continuous readings of the transvesical pressures using LabChart 7.3 Software (AD Instruments). Compliance was calculated as the percentage of bladder filling at pressures less than 20 cm H₂O. Leak point pressure was noted to be the maximum pressure attained at terminal contraction.

Statistical analysis. The statistical differences between control and treatment groups for urothelial width, muscle quantification, vessel quantification, bladder capacity, and nerve length were calculated by a two-sample t-test. The two-sample t-test tests the null hypothesis that the means of each continuous variable measured from both the control and experimental groups are equal and provides a measure of statistical significance (p value). A 95% confidence interval (CI) was utilized to estimate the mean of the obtained data points. A p value <0.05 was considered statistically significant. Standard error was calculated dividing the standard deviation by the sample size's square root.

1. A method comprising administering a MSC/CD34+ HSPC co-cultured TCM to a subject.

2. The method of claim 1, wherein the MSC/CD34+ HSPC co-cultured TCM comprises substances selected from the group consisting of: cytokines, chemokines, anti-inflammatory factors, growth factors, hormones, and/or extracellular matrix components.

3. The method of claim 1, wherein the subject suffers from damage to a tissue, trauma to a tissue, and/or loss of a tissue.

4. The method of claim 1, wherein the MSC/CD34+ HSPC co-cultured TCM is administered to the damaged tissue and/or to the traumatized tissue.

5. The method of claim 1, further comprising providing a graft to a subject.

6. The method of claim 5, wherein the MSC/CD34+ HSPC co-cultured TCM is administered after the graft.

7. The method of claim 1, wherein administering comprises instilling the MSC/CD34+ HSPC co-cultured TCM into the bladder of the subject.

8. The method of claim 7, wherein instilling comprises filling the bladder with a solution and holding the solution for a period of time.

9. The method of claim 8, wherein the period of time is selected from the group consisting of: 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, and 30 minutes.

10. The method of claim 1, further comprising repeating the administration.

11.-12. (canceled)

13. The method of claim 1, wherein the percent vasculature of the blood vessels observed in tissue following administration is higher than the percent vasculature of the blood vessels observed in tissue without administration.

14. The method of claim 8, further comprising increasing the solution volume per week to a final volume as determined by the volume at which urethral leakage occurs and continuing the administration for a period of more than one week.

15. The method of claim 1, wherein the MSC/CD34+ HSPC co-cultured TCM is administered in a dosage unit formulation selected from the group consisting of: conventional non-toxic pharmaceutically acceptable carriers, adjuvants, and vehicles.

16. The method of claim 3, wherein the tissue is selected from the group consisting of: epithelial tissue, fibrous tissue, cartilage tissue, bone tissue, blood vessels, muscle tissue, and nerve tissue.

17. The method of claim 1, wherein the subject suffers from bladder disease.

18. The method of claim 17, wherein the bladder disease is selected from the group consisting of: cystitis, interstitial cystitis, overactive bladder, bladder cancer, bladder stones, and bladder prolapse.

19. A method comprising (1) administering to the subject a first therapy comprising providing a tissue graft to a subject and, thereafter, administering to the subject a composition comprising a MSC/CD34+ HSPC co-cultured TCM and (2) administering to the subject a second therapy.

20. The method of claim 19, wherein the second therapy is regenerative therapy selected from the group consisting of: cell therapy, immunomodulation therapy, tissue engineering, viscosupplementation, and prolotherapy.

21. The method of claim 19, wherein the second therapy is selected from the group consisting of: blood tests, imaging tests, physical exams, and/or iodine uptake tests.

22. The method of claim 5, wherein the graft is selected from the group consisting of: a seeded or an unseeded graft, one or more cells on a scaffold, and tissue.
